(19) United States
(12) Patent Application Publication
(10) Pub. No.: US 2025/0348640 A1
(43) Pub. Date: Nov. 13, 2025

(54) Computer Method to Optimize Contrast-Agent Volume and Injection Force for Vascular Imaging

(71) Applicant: LightLab Imaging, Inc., Westford, MA (US)

(72) Inventors: Dhananjay Radhakrishnan Subramaniam, Beachwood, OH (US); Wei C. Chiu, Acton, MA (US)

(73) Assignee: LightLab Imaging, Inc., Westford, MA (US)

(21) Appl. No.: 19/203,620

(22) Filed: May 9, 2025

Related U.S. Application Data
(60) Provisional application No. 63/645,269, filed on May 10, 2024.

(51) Int. Cl. G06F 30/28; G06F 113/08; G06T 7/00

(52) U.S. Cl. CPC G06F 30/28; G06T 7/0012; G06F 2113/08; G06T 2207/10101

(57) ABSTRACT

Systems and methods described herein relate to developing a virtual anatomical model of a coronary artery with specific conditions and related fluid properties, such as the velocity and viscosity of selected fluid, to provide accurate parameters for an intravascular imaging procedure. The resulting parameters may relate to the contrast agent volume, time for pullback of intravascular tool, and contrast agent injection force. The models may be patient specific, based on characteristics of a patient's artery. The systems and methods may be utilized for PCI planning, vascular device design and process optimization.

### CROSS-REFERENCE TO RELATED APPLICATION

This application claims the benefit of the filing date of U.S. Provisional Patent Application No. 63/645,269, filed on May 10, 2024, the disclosure of which is hereby incorporated herein by reference.

### BACKGROUND

Functional and anatomical assessment of coronary arteries, specifically vascular lesions within the arteries, can be aided by in-vitro bench testing. In-vitro bench testing can subsequently assist clinicians to develop percutaneous therapeutic strategies. Idealized artery models fabricated from polymeric material could be filled with contrast to visualize the positioning of a stent. Such in-vitro models assist in evaluating different vascular prosthesis and aid in validating quantitative angiography systems. However, in-vitro testing is costly and labor intensive. Moreover, the rigid polymeric coronary artery models filled with contrast agent do not directly mimic the human anatomy.

Alternatively, virtual angiograms applying a virtual-ink computational fluid dynamics (“CFD”) method are used to determine the contrast-agent concentration necessary for intravascular imaging procedures. The virtual-ink method assumes that the two fluids (i.e., blood and contrast agent) have equivalent viscosities. However, contrast agents used by clinicians can have viscosities that are between 23% to 530% higher than the viscosity of blood. Furthermore, the virtual-ink method assumes that advection of the contrast agent occurs at the same velocity as that for the blood. Conversely, in a clinical setting, the contrast-agent velocity could be significantly different than the blood velocity.

### BRIEF SUMMARY

Systems and methods described herein may generate virtual anatomical model with conditions more closely related to real world properties, such as the velocity and viscosity of selected fluid, to provide more accurate parameters for intravascular imaging procedure. The parameters may relate to the contrast agent volume, time for pullback of intravascular tool, and contrast agent injection force. The models may be patient specific, based on characteristics of a patient's artery. These systems and methods may be utilized for percutaneous coronary intervention (“PCI”) planning, vascular device design, and process optimization.

One aspect of the technology is directed to a method comprising receiving anatomical characteristics of a coronary artery, generating, based on the anatomical characteristics of the coronary artery, a virtual model, wherein the virtual model includes a region of interest comprising a proximal end and a distal end, receiving a first fluid and a second fluid, wherein the first fluid has a first velocity and a first viscosity value, wherein the second fluid has a second velocity profile and a second viscosity value. The method may further comprise simulating, using a computational fluid dynamics model using the first viscosity value and the second viscosity value, a flow of the first fluid and the second fluid through the virtual model, wherein the computational fluid dynamics model has boundary conditions including at least the first velocity profile and the second velocity profile. The method may further comprise calculating, using the computational fluid dynamics model, a proximal volume fraction of the first fluid at the proximal end and a distal volume fraction and calculating based on the first velocity and the flow of the first fluid, a flow rate profile of the first fluid. The method may use the proximal volume fraction, the distal fraction volume fraction, and the flow rate profile, a recommendation for a volume of the first fluid. In some examples, the computational fluid dynamics model may simulate a simultaneous flow of the first fluid and the second fluid. The flow rate profiles may be a function of the first fluid with the virtual model and time.

In some examples, the method may further comprise setting, based on the recommendation, a parameter for the volume of the first fluid to be injected in the coronary artery. In some examples, the method may further comprise measuring a time for the first fluid to flow from the proximal end to the distal end of the virtual model and calculating, using the computational fluid dynamics model a time frame available for a pullback of an imaging tool, wherein the time frame is derived from a comparison of a function of the proximal volume fraction by the measured time and the second function of the distal volume fraction by the measured time. The method may further comprise setting, based on the time available for the pullback, a pullback time for the imaging tool.

In some examples, the recommendation for the volume of the first fluid corresponds to an area under a curve of the flow rate profile. The method may further comprise calculating, using the computational fluid dynamics model, an injection profile for the first fluid derived from a function of injection force of the first fluid by time.

The virtual model may have a first fluid inlet, a second fluid inlet, an outlet, and a wall. The boundary conditions further include no slip condition applied to the wall of the virtual model. The boundary conditions may further include a pressure profile applied to the outlet of the virtual model.

Another aspect of the technology is directed to a system comprising one or more processors, wherein the one or more processors are configured to receive anatomical characteristics of a coronary artery, generate, based on the anatomical characteristics of the coronary artery, a virtual model, wherein the virtual model includes a region of interest comprising a proximal end and a distal end, receive a first fluid and a second fluid, wherein the first fluid has a first velocity profile and a first viscosity value and the second fluid has a second velocity profile and a second viscosity value, simulate, using a computational fluid dynamics model using the first viscosity value and the second viscosity value, a flow of the first fluid and the second fluid through the virtual model, wherein the computational fluid dynamics model has boundary conditions including at least the first velocity profile and the second velocity profile. The one or more processors may be configured to further calculate, using the computational fluid dynamics model, a proximal volume fraction and the first fluid at the proximal and a distal volume fraction of the first fluid at the distal end and calculate using the computational fluid dynamics model based on the first velocity profile and the flow of the first fluid, a flow rate profile of the first fluid. The system may output, based on the proximal volume fraction, the distal volume fraction, and the flow rate profile, a recommendation for the volume of the first fluid.

In some examples, the one or more processors are further configured to set, based on the recommendation, a parameter for the volume of the first fluid to be injected into the coronary artery. The one or more processors may be further configured to measure a time for the first fluid to flow from the proximal end to the distal end of the virtual model, calculate, using the computational fluid dynamics model, a time frame available for a pullback of an imaging tool. The time frame may be derived from a comparison of a first function of the proximal volume fraction by the measured time and a second function of the distal volume fraction by the measured time. The one or more processors may be further configured to set, based on the time frame available for the pullback, a pullback time for the imaging tool.

In some examples, the recommendation for the volume of the first fluid corresponds to an area under a curve of the flow rate profile. The one or more processors may be further configured to calculate, using the computational fluid dynamics model, an injection profile for the first fluid derived from a function of injection force of the first fluid by time.

The virtual model may have a first fluid inlet, a second fluid inlet, an outlet, and a wall. The boundary conditions may further include no slip condition applied to the wall of the virtual model. The boundary conditions may further include a pressure profile applied to the outlet of the virtual model.

Yet another aspect of the technology is directed to one or more non-transitory computer storage media encoding instructions that, when executed by one or more processors, cause the one or more processors to perform the operations comprising receiving anatomical characteristics of a coronary artery, generating, based on the anatomical characteristics of the coronary artery, a virtual model, wherein the virtual model includes a region of interest comprising a proximal end and a distal end, receiving a first fluid and a second fluid, wherein the first fluid has a first velocity and a first viscosity value, wherein the second fluid has a second velocity profile and a second viscosity value. The instructions may further encode for simulating, using a computational fluid dynamics model using the first viscosity value and the second viscosity value, a flow of the first fluid and the second fluid through the virtual model, wherein the computational fluid dynamics model has boundary conditions including at least the first velocity profile and the second velocity profile. The instructions may further encode for calculating, using the computational fluid dynamics model, a proximal volume fraction of the first fluid at the proximal end and a distal volume fraction and calculating based on the first velocity and the inlet area of the first fluid, a flow rate profile of the first fluid. The method may use the proximal volume fraction, the distal fraction volume fraction, and the flow rate profile, a recommendation for a volume of the first fluid.

In some examples, the one or more non-transitory computer storage media may further comprise setting, based on the recommendation, a parameter for the volume of the first fluid to be injected in the coronary artery. In some examples, the one or more non-transitory computer storage media may further comprise measuring a time for the first fluid to flow from the proximal end to the distal end of the virtual model and calculating, using the computational fluid dynamics model a time frame available for a pullback of an imaging tool, wherein the time frame is derived from a comparison of a function of the proximal volume fraction by the measured time and the second function of the distal volume fraction by the measured time. The one or more non-transitory computer storage media may further comprise setting, based on the time available for the pullback, a pullback time for the imaging tool.

In some examples, the recommendation for the volume of the first fluid corresponds to an area under a curve of the flow rate profile. The instructions may further comprise calculating, using the computational fluid dynamics model, an injection profile for the first fluid derived from a function of injection force of the first fluid by time.

The virtual model may have a first fluid inlet, a second fluid inlet, an outlet, and a wall. The boundary conditions further include no slip condition applied to the wall of the virtual model. The boundary conditions may further include a pressure profile applied to the outlet of the virtual model.

### BRIEF DESCRIPTION OF THE DRAWINGS

FIG. 1 is a system of schematic diagram of an example flow simulation system according to aspects of the disclosure.

FIG. 2 is an illustration of a virtual model including a region of interest, according to aspects of the disclosure.

FIG. 3 is a block diagram of an example flow simulation system according to aspects of the disclosure.

FIG. 4A is an illustration of the boundary conditions set at the inlet of the virtual model according to aspects of the disclosure.

FIG. 4B is an illustration of the boundary conditions at the outlet of the virtual model according to aspects of the disclosure.

FIG. 5A is an illustration of the time-varying contrast-agent volume fraction for the proximal end and distal end of the region of interest according to aspects of the disclosure.

FIG. 5B is an illustration of example flow rate profiles output from a virtual model according to aspects of the disclosure.

FIG. 6A is a graphical representation of the time-varying contrast-agent volume fraction for the proximal end and the distal end of the region of interest according to aspects of the disclosure.

FIG. 6B is a graphical representation of the force-time profile for the inlet of the virtual model, according to aspects of the disclosure.

FIG. 7 is a flow chart of an embodiment of the method of outputting a volume fraction for a first fluid according to aspects of the disclosure.

FIGS. 8 and 9 are illustrations of the time-varying contrast-agent volume fraction for the proximal end and distal end of a region of interest for a subject-specific analysis according to aspects of the disclosure.

FIGS. 10 and 11 are graphical representations of the time-varying contrast-agent volume fraction for the proximal end and the distal end of a region of interest for a subject-specific analysis according to aspects of the disclosure.

### DETAILED DESCRIPTION

Systems and methods described herein may perform coronary artery modelling and flow simulation using computational fluid dynamics methodology. For example, the systems and methods may construct a virtual anatomical model of a coronary artery and simulate the flow of multiple fluids through the model under various conditions. In some examples, the conditions may relate to specific properties of the fluids in the simulation, such as viscosities and velocities. In some examples, the simulations may be run to ascertain the optimal parameters for imaging a coronary artery. For example, model-predicted volume fractions may be used to determine the time allotted for a pullback of an intravascular imaging tool or the optimal injection force for the contrast agent. Further, in some examples, the model-predicted flow rate may be used to determine the optimal amount of a contrast agent to use. The systems and methods disclosed herein may have multiple practical uses including vascular device design, e.g., estimation of pullback parameters, and process optimization, e.g., bolus injection parameters for blood-flow measurement.

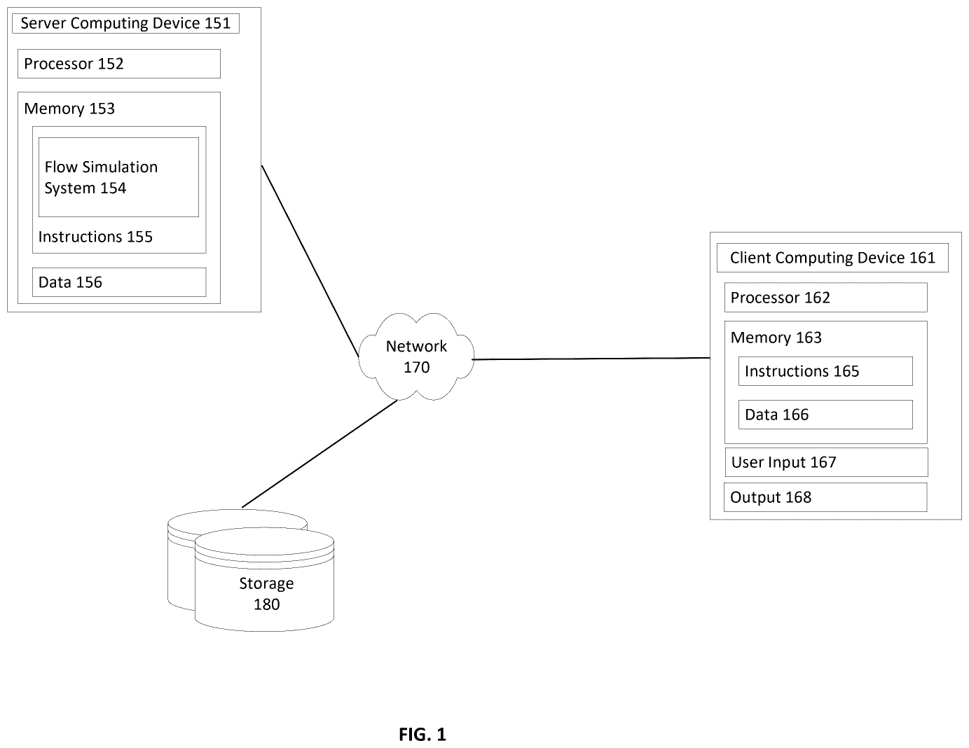

FIG. 1 depicts a block diagram of an example environment for implementing a flow simulation system. The flow simulation system can be implemented on one or more devices having one or more processors in one or more locations, such as in server computing device. Client computing device 161 and the server computing device 151 can be communicatively coupled to one or more storage devices 180 over a network 170. The storage devices 180 can be a combination of volatile and non-volatile memory and can be at the same or different physical locations than the computing devices. For example, the storage devices 180 can include any type of non-transitory computer readable medium capable of storing information, such as a hard-drive, solid state drive, tape drive, optical storage, memory card, ROM, RAM, DVD, CD-ROM, write-capable, and read-only memories.

The server computing device 151 can include one or more processors 152 and memory 153. The memory 153 can store information accessible by the processors 152, including instructions 155 that can be executed by the processors 152. The memory 153 can also include data 156 that can be retrieved, manipulated, or stored by the processors 152. The memory 153 can be a type of non-transitory computer readable medium capable of storing information accessible by the processors 152, such as volatile and non-volatile memory. The processors 152 can include one or more central processing units (CPUs), graphic processing units (GPUs), field-programmable gate arrays (FPGAs), and/or application-specific integrated circuits (ASICs), such as tensor processing units (TPUs).

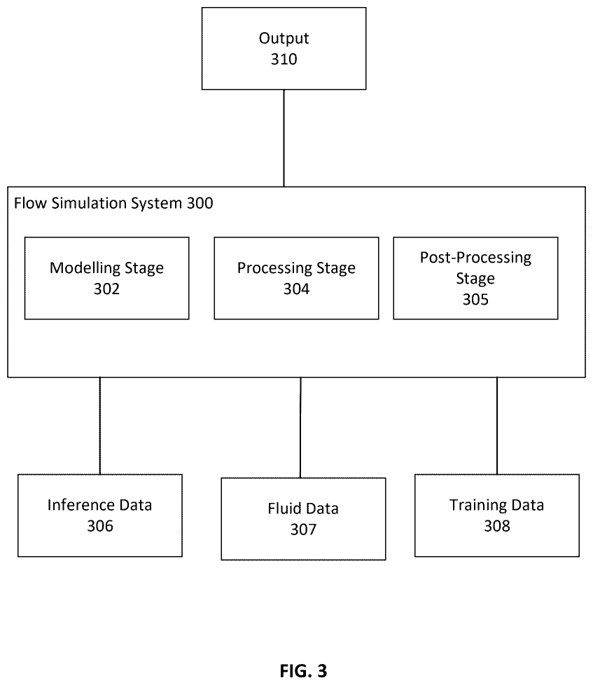

The instructions 155 can include one or more instructions that, when executed by the processors 152, cause one or more processors 152 to perform actions defined by the instructions 155. The instructions 155 can be stored in object code format for direct processing by the processors 152, or in other formats including interpretable scripts or collections of independent source code modules that are interpreted on demand or compiled in advance. The instructions 155 can include instructions for implementing a flow simulation system 154, which can correspond to the flow simulation system of FIG. 3. The flow simulation system 154 can be executed using the processors 152, and/or using other processors remotely located from the server computing device.

The data 156 can be retrieved, stored, or modified by the processors 152 in accordance with the instructions 155. The data 156 can be stored in computer registers, in a relational or non-relational database as a table having a plurality of different fields and records, or as JSON, YAML, proto, or XML documents. The data 156 can also be formatted in a computer-readable format such as, but not limited to, binary values, ASCII, or Unicode. Moreover, the data 156 can include information sufficient to identify relevant information, such as numbers, descriptive text, proprietary codes, pointers, references to data stored in other memories, including other network locations, or information that is used by a function to calculate relevant data.

The client computing device 161 can also be configured similarly to the server computing device 151, with one or more processors 162, memory 163, instructions 165, and data 166. The client computing device 161 can also include a user input 167 and an output 168. The user input 167 can include any appropriate mechanism or technique for receiving input from a user, such as keyboard, mouse, mechanical actuators, soft actuators, touchscreens, microphones, and sensors.

The server computing device 151 can be configured to transmit data to the client computing device 161 over network 170. The client computing device 161 can be configured to display at least a portion of the received data 166 on a display implemented as part of the output 168. The output 168 can also be used for displaying an interface between the client computing device 161 and the server computing device 151. The output 168 can alternatively or additionally include one or more speakers, transducers or other audio outputs, a haptic interface or other tactile feedback that provides non-visual and non-audible information to the platform user of the client computing device.

Although FIG. 1 illustrates the processors and the memories as being within the computing devices, components described herein can include multiple processors and memories that can operate in different physical locations and not within the same computing device. For example, some of the instructions and the data can be stored on a removable SD card and others within a read-only computer chip. Some or all of the instructions and data can be stored in a location physically remote from, yet still accessible by, the processors. Similarly, the processors can include a collection of processors that can perform concurrent and/or sequential operation. The computing devices can each include one or more internal clocks providing timing information, which can be used for time measurement for operations and programs run by the computing devices.

The devices can be capable of direct and indirect communication over the network 170. The network 170 itself can include various configurations and protocols including the Internet, World Wide Web, intranets, virtual private networks, wide area networks, local networks, and private networks using communication protocols proprietary to one or more companies. The network 170 can support a variety of short-and long-range connections. The short-and long-range connections may be made over different bandwidths, such as 2.402 GHz to 2.480 GHz, commonly associated with the Bluetooth® standard, 2.4 GHz and 5 GHz, commonly associated with the Wi-Fi® communication protocol; or with a variety of communication standards, such as the LTE® standard for wireless broadband communication. The network 170, in addition or alternatively, can also support wired connections between the devices and the data center, including over various types of Ethernet connection.

Although a single server computing device, client computing device, and storage are shown in FIG. 1, it is understood that the aspects of the disclosure can be implemented according to a variety of different configurations and quantities of computing devices, including in paradigms for sequential or parallel processing, or over a distributed network of multiple devices. In some implementations, aspects of the disclosure can be performed on a single device connected to hardware accelerators configured for processing optimization models, and any combination thereof.

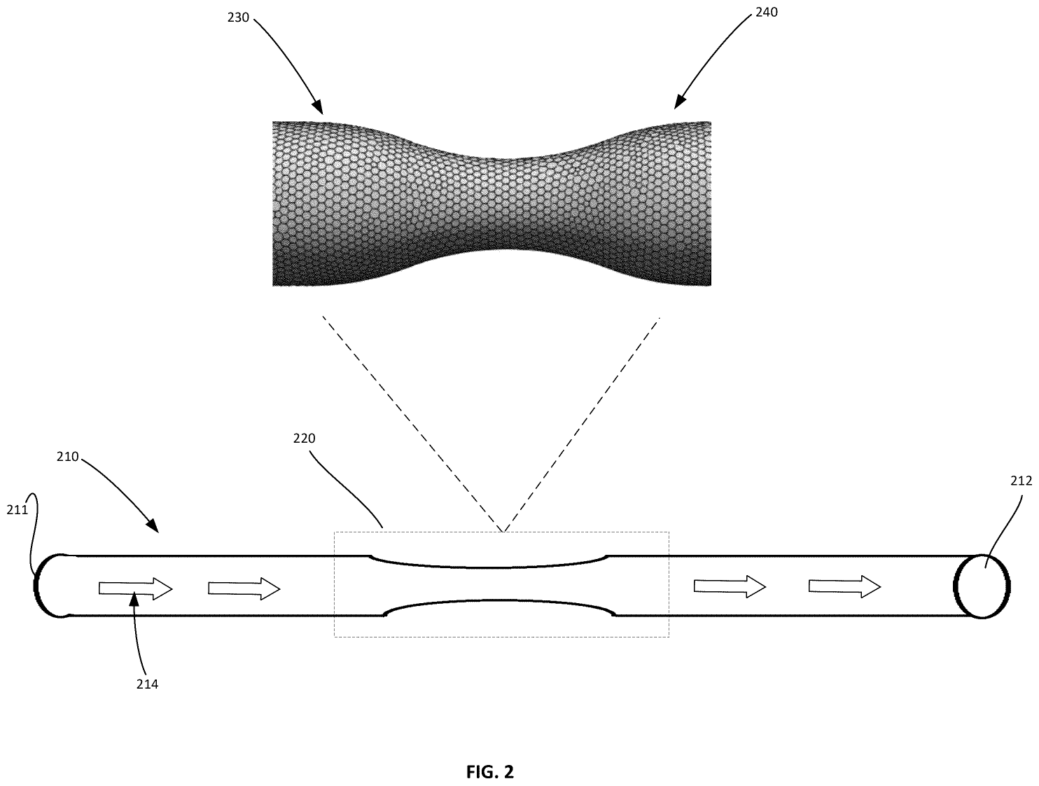

FIG. 2 illustrates a virtual anatomical model of an idealized coronary artery generated by a flow simulation system as described herein. The model 210 may be developed using the three-dimensional geometry of a coronary artery. In some examples, the model 210 may be constructed using specialized software, such as three-dimensional computer aided design engineering software. In some examples, software may be configured to receive image data gathered using imaging techniques, such as optical coherence tomography (“OCT”), intravascular ultrasound (“IVUS”), near infrared spectroscopy (“NIRS”), optical frequency domain imaging (“OFDI”), or any other imaging technique to image a coronary artery. For example, image data of a coronary artery may be input into the software to replicate or generate a the three-dimensional (“3D”) model of the imaged coronary artery. Some of the imaging techniques require the use of catheters to administer a contrast agent or deliver an imaging tool to an area of the coronary artery. To replicate results of a real-world percutaneous intervention (“PCI”), the software may generate models of catheters, such as guiding and imaging catheters, and integrate the model catheters within the idealized artery. The catheter models may be integrated with the artery model using Boolean operations. In some examples, the model 210 may be an artery-catheter assembly formed by integrating the virtual catheter and artery.

The model 210 includes an inlet 211 and an outlet 212. Relative to the directional flow 214 of the model 210, the inlet 211 is upstream on the idealized artery and the outlet is downstream. The directional flow 214 may relate to the flow of the fluid within the model 210. The model 210 also includes a wall. The wall defines the outer bounds of the model 210, such that in the fluid simulator the fluid would flow through the confines of the wall. The wall may relate to the wall of the artery model, the wall of the catheter model or the wall of the artery-catheter assembly.

Within the fluid simulation software, boundary conditions may be set for specific portions of the model 210, such as the inlet 211, outlet 212, and the wall. The boundary conditions are a set of constraints to boundary value problems in computational fluid dynamics. For example, a no-slip condition may be specified at the wall of model 210, to ensure that within the simulation the fluid adheres to the walls of the artery and the catheter.

The model 210 further includes a region of interest (“ROI”) 220. The ROI 220 may correspond to an area of the model 210 with varied characteristics from the rest of the idealized artery. For example, the ROI 220 may correspond to a stenosis with a narrowed diameter. The ROI 220 includes a proximal end 230 and a distal end 240. In some examples, the ROI may be manually selected by a user or automatically detected by the system. In some examples, there may be multiple ROIs on a model, each having a distinct proximal and distal end.

The contrast-agent concentration, contrast agent injection volume, and contrast agent injection rate or force are optimized to increase the quality of the intravascular images obtained during the procedure. Advanced models of flow dynamics enable the physiological significance of lesions to be estimated under both normal and hyperemic conditions. Furthermore, computational fluid dynamics (“CFD”) models can be used to simulate multiphase flows involving blood and contrast agent. CFD models may use a numerical Navier-Stokes solver or Lattice-Boltzmann differential equations of fluid flow to calculate values for aspects of the model. For example, a Navier-Stokes solver may be used to calculate the stenotic resistance in a model. The vessel contours are delineated by OCT and the flow within the walls is broken into thousands of small volumes. Simultaneously, at each volume, the Navier-Stokes momentum and conservation of mass equations are solved to compute the flow field through the volume. From this flow field the pressure drop along the vessel is found.

FIG. 3 depicts a block diagram of an example flow simulation system 300 to be implemented as described in connection with FIG. 1. The system 300 may be configured to run dynamic fluid simulations on virtual artery-catheter models. The system 300 may be executed in three stages, including a modelling stage 302, a processing stage 304, and a post-processing stage 305. The stages can be performed in sequence or at least partially in parallel. Each stage may utilize fluid simulation software or open-source tools.

The modelling stage 302 may be configured to generate CFD models of the artery-catheter assemblies. In some examples, the artery-catheter assembly formed by integrating the virtual catheter and artery may be exported to system 300. The modelling stage 302 may be configured to receive inference data 306 and/or training data 308 or to use inference data 306 and/or training data 308 to generate the 3D virtual models.

The modelling stage 302 may be configured to create an unstructured tetrahedral mesh of the computational domain, using inference data 306 and/or the training data 308. The tetrahedral mesh may be combined to create polyhedral elements, such as the 3D shape depicted in FIG. 2. The polyhedral elements are preferable for running blood-flow simulations in the system 300. The modelling stage 302 may be an AI model, in some examples.

In some examples, the inference data 306 may be patient specific image data of an artery. For example, the inference data 306 may include intraluminal images of a patient, extraluminal images of a patient, other health related factors associated with the patient, or the like. The inference data 306 may also be generalized data collected from prior procedures or prior simulations relating to an idealized artery for training purposes.

The training data 308 may include consecutive intravascular images, e.g., consecutive image frames captured during a pullback of an imaging tool. According to some examples, the intravascular images may be stacked. In some examples, the training data 308 may be 3D data. The 3D data may be three-dimensional images. In some examples, the 3D images are generated based on intravascular imaging data, such as intravascular images. For example, intravascular images captured by an intravascular imaging tool during a pullback may be used to generate 3D images. In some examples, the intravascular images captured during the pullback may be chunked, or grouped, into sections of frames. The 3D images may be generated based on the sections of frames.

The modelling stage 302 may be further configured to apply specific conditions, such as boundary conditions, to the artery-catheter models. For example, the system 300 may receive boundary conditions for specific aspects of the model. The boundary conditions may include inlet boundary conditions, outlet boundary conditions, wall boundary conditions, constant boundary conditions, axisymmetric boundary conditions, symmetric boundary conditions, and periodic or cyclic boundary conditions. For example, a no-slip condition may be specified at the wall of the model, to ensure that within the simulation the fluid adheres to the walls of the artery and the catheter.

The modelling stage 302 may be configured to receive fluid data 307. The fluid data 307 may relate to information about the fluid selected to be run through the system 300, such as the number of fluids to be run, the types of fluids, the properties of the fluids, including thermophysical properties, etc. Within the fluid data 307, multiple different fluids may be selected for flow simulation. For example, the fluid data 307 may include the selection of a contrast agent as one fluid and human blood as the second fluid. The thermophysical properties may include dynamic viscosity, density, enthalpy, entropy, heat capacity, thermal conductivity, or dielectric constant. At the modelling stage 302, the system 300 may assume the fluids adhere to Newtonian behavior and are incompressible. In some examples, the system 300 may introduce non-Newtonian effects in the model to more accurately model the rheology of the selected fluids.

The fluid data 307 may include information regarding the viscosity of the selected fluids. Viscosity is a thermophysical constant, which can be measured using an instrument known as a viscometer. Current, virtual fluid simulation systems, such as virtual ink, assume that blood and contrast agent have the same viscosities. However, contrast agents used by clinicians can have viscosities that are between 23% to 530% higher than the viscosity of blood. This issue lends to inaccurate results that are not immediately applicable to intravascular imaging procedures. Without taking into account the varying viscosities of the fluids, a technician may have to manually alter results from the simulation or repeatedly inject an amount of fluid, i.e. contrast agent, into a patient until a quality intravascular image can be captured.

The fluid data 307 may be manually input by a user of the system 300. In some examples, the user input may be properties of the fluids to be populated into the fluid data 307. For example, if contrast agent X is selected as a fluid for flow simulation, the properties associated with contrast agent X, such as the dynamic viscosity and density, may be included in the fluid data 307. In some examples, properties of selected fluid may be the result of an aggregate of data collected from previous procedures or simulations. For example, velocity profiles may be generated for a selected fluid based on the data collected from prior simulations or in-vivo measurements and can be subsequently used to generate an average velocity profile for the selected fluid. Existing virtual flow simulation methods, including the virtual-ink method, assume that the advection of the contrast agent occurs at the same velocity as that for the blood. However, the contrast agent velocity could be significantly different than the blood velocity. By taking these differences into account, the flow simulation system 300 generates simulations more similar to its clinical counterpart and more accurate simulation results.

At modelling stage 302, the fluid data 307 may be applied to the artery-catheter model with boundary conditions. For example, based on the fluid data 307, velocity profiles assigned to the selected fluid may be prescribed as inlet boundary conditions. Based on the fluid data 307, a pressure profile may be prescribed as outlet boundary conditions. The velocity and pressure profiles of selected fluids may be generated based on prior simulations or previous PCIs. For example, if contrast agent X is selected as a fluid, the modelling stage 302 may be configured to refer to previous simulations using contrast agent X, specifically, using the time required for contrast agent X to travel over a defined distance, to generate a velocity profile of contrast agent X. In some examples, the velocity and pressure profiles may be based on properties of the selected fluid and the artery-catheter model. For example, if contrast agent X is selected as a fluid, the cross-sectional area of the artery-catheter model and the known density of contrast agent X may be utilized to build a velocity profile for contrast agent X.

The processing stage 304 may be configured to utilize a free-surface method to simulate the simultaneous flow of selected fluids through the artery-catheter model with the applied boundary conditions set. The simulation may be an injection simulation wherein one fluid is injected into the other fluid within the artery-catheter model. For example, the processing stage 304 may run a simulation where contrast agent X is injected into the artery-catheter model, wherein the model has a constant flow of blood. In some examples, the processing stage 304 may utilize a volume-of-fluid (“VOF”) method to simulate the flow of the selected fluids. In CFD, the VOF method is a free-surface modelling technique, i.e. a numerical technique for tracking and locating the free surface. The VOF method is well suited for simulating core-annular flows and capturing the interaction between at least two fluids in a multiphase flow.

The processing stage 304 may be configured to capture and record data from a simulation of the selected fluids through the virtual artery-catheter model. In some examples, the processing stage 304 may yield cross-sectional images of the ROI during the simulation. For example, time stamped images of a slice of the ROI may be captured depicting the selected fluids interacting with each other during the injection simulation. These images may depict the amount of fluid present at that slice of the ROI at any time during the simulation. These images may be used to calculate the volume fraction of a fluid at a time during the simulation. The volume fraction is used to quantify the amount of fluid needed to be injected into the coronary artery. For example, where a first fluid is contrast agent X and a second fluid is blood within the artery, the volume fraction may be utilized to determine how much contrast agent X is needed to clear the blood from the artery for optimal intravascular imaging.

The post-processing stage 305 may be configured to use visualization software to process the results from the processing stage 304. In some examples, the visualization software may be specialized software, such as an open-source, multi-platform data analysis and visualization application. The post-processing stage 305 may include steps such as determining phase or volume fraction of the proximal end and the distal end of the ROI of the artery-catheter model.

The post-processing stage 305 may quantify the volume fraction of the proximal and distal end of the ROI at various times throughout the simulation. In some examples, the post-processing stage 305 may be configured to plot the volume fraction across the time of the injection simulation. In some examples, the post-processing stage 305 may generate time plots where the volume fraction is plotted against the time in seconds for the first fluid to flow through ROI of the artery-catheter model.

In some examples, the post-processing stage 305 may be configured to plot the force within the artery-catheter model against the time in seconds to yield a force-time profile. This profile may be utilized to determine the amount of force necessary to effectively inject a fluid into the artery.

The flow simulation system 300 may be configured to analyze and compare the output of modelling stage 302, the output of processing stage 304 and the output of post-processing stage 305. For example, the flow simulation system 300 may compare the area of the artery-catheter model generated at the modelling stage 302 to the volume fraction results of the processing stage 304 and post-processing stage 305 to generate an indication as to the necessary amount of a selected fluid required for optimal imaging visualization within the artery. The output 310 of the flow simulation system 300 may indicate an optimal volume of fluid to be injected.

In some examples, the optimal volume determined may correspond to contrast agents. While contrast agents improve visibility of necessary internal organs and vascular systems, overuse of contrast agents may lead to an increased risk of allergic reactions, contrast-induce nephropathy, renal failure, thyroid disfunction, or other life-threatening emergencies. By determining, in the planning phase with flow simulations on patient specific models, the optimal amount of contrast agent required to obtain quality intravascular images, the risks associated with overuse of contrast agent are lessened. Based on the optimal volume of contrast agent determined, intravascular images may be obtained more efficiently and with less risk to the patient.

The flow simulation system 300 may be configured to analyze and compare the output of modelling stage 302, the output of processing stage 304 and the output of post-processing stage 305. For example, the flow simulation system 300 may compare the area of the artery-catheter model generated at the modelling stage 302 to the volume fraction results of the processing stage 304 and post-processing stage 305 to determine the optimal pullback speed of an intravascular tool during an PCI or intravascular imaging procedure. In some examples, the flow simulation system 300 may be configured to compare the artery-catheter model generated in the modelling stage 302 and the results from the post-processing stage 305 to determine the time available for the pullback of the intravascular imaging tool.

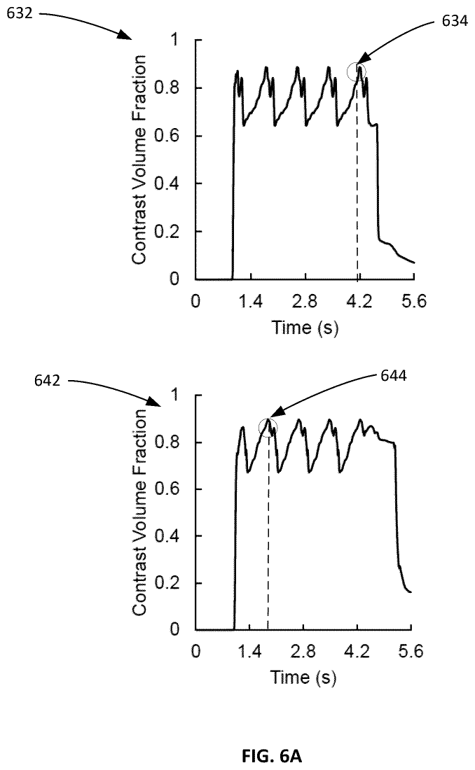

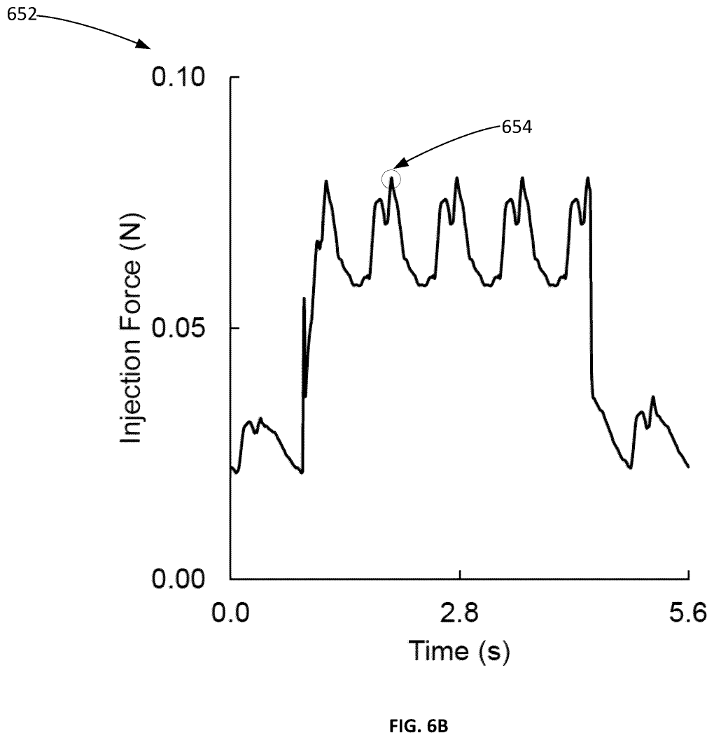

Explained in more detail in relation to FIG. 6A, the system 300 may compare volume fraction time plots of the proximal and distal ends of the ROI to determine the amount of time available with a high-volume fraction to perform the pullback. This determined available time, along with the length of the ROI, may be used to determine the optimal pullback speed of the intravascular tool. The output 310 of the flow simulation system 300 may be a time available for the pullback and optimal pullback speed of an intravascular imaging tool. The ability to determine the pullback time and speed prior to the intravascular imaging reduces the time a technician may spend performing the imaging procedure on a patient and the number of pullbacks required to obtain a quality image of the ROI.

The flow simulation system 300 may be configured to analyze and compare the output of modelling stage 302, the output of processing stage 304 and the output of post-processing stage 305. For example, the flow simulation system 300 may compare the area of the artery-catheter model generated at the modelling stage 302 to the volume fraction results of the processing stage 304 and post-processing stage 305 to generate the optimal force required for the injection of the fluid into the artery. In some examples, the flow simulation system 300 may compare the processing stage results with the area of the selected fluid at the inlet of the model and the pressure exerted on the fluid at the inlet to determine required force. The output 310 of the flow simulation system 300 may indicate an optimized force of injection for a fluid to increase the quality of the intravascular images obtained during an imaging procedure.

The output 310 of the flow simulation system 300 may be input into an imaging system such that the output 310 are applied to the imaging procedure. In some examples, if output 310 includes an optimal volume of the contrast agent and a pullback speed for the intravascular imaging probe, that output may be received at the imaging system and applied as parameters to the imaging procedure. By inputting these parameters directly in the imaging system, potential errors during the procedure may be reduced. Further, the imaging procedure may be streamlined and more efficient for having these patient specific parameters previously tested using the simulation system.

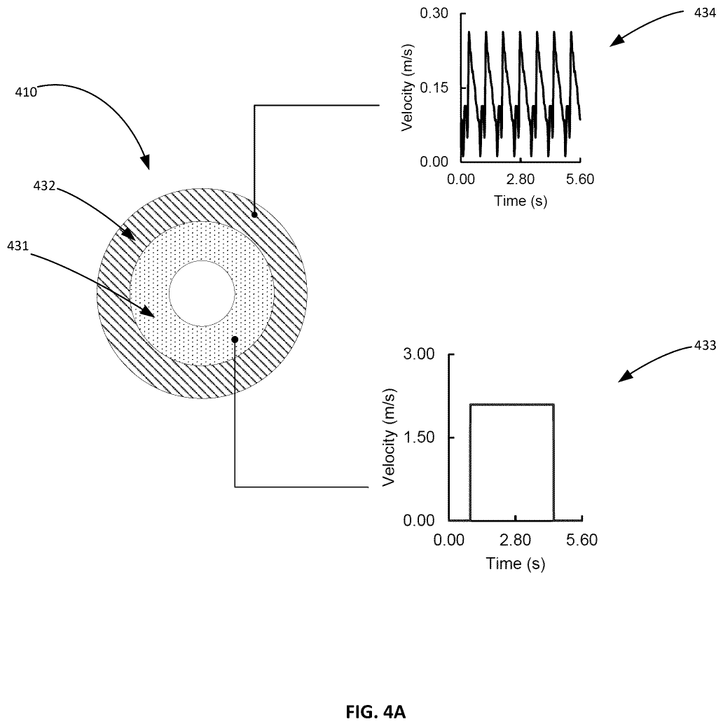

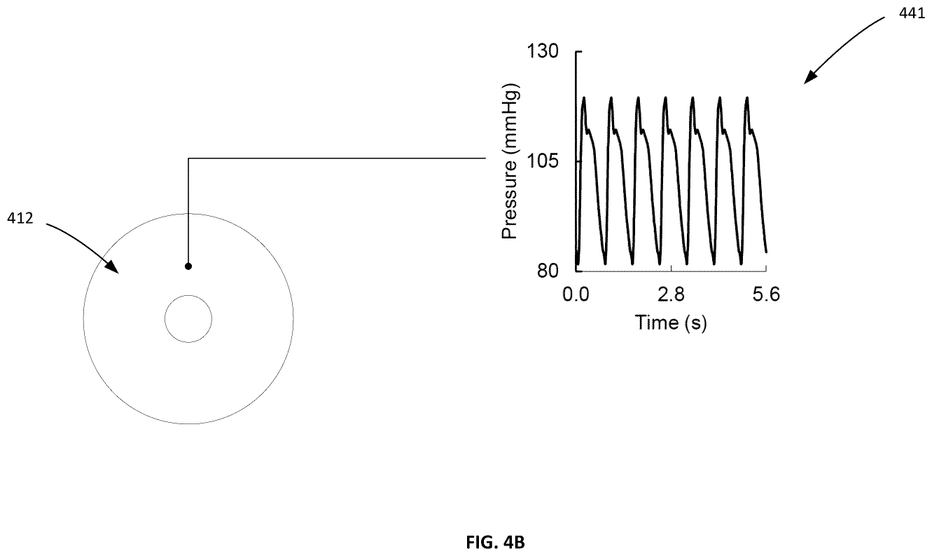

FIG. 4A-4B illustrate example boundary conditions set within the fluid simulation system for a virtual model. In FIG. 4A, model 410 depicts the inlet boundary conditions are set within the software at inlet 430 of the artery-catheter model. Specifically, the system may receive two fluids, a first fluid 431 and a second fluid 432, for simulation to flow through the virtual model. At the inlet 430, the two fluids may be concentrically aligned, such that the first fluid 431 flows concentrically within the second fluid 432. In some examples, there may be more than two fluids present within the virtual model. In some examples, there may be only one fluid present within the model. The first fluid 431 may correspond to a contrast to be injected into the artery. In some examples, the second fluid 432 may correspond to blood in the artery.

The fluid simulation system may receive properties, such as thermophysical properties, of the first fluid 431 and the second fluid 432. In some examples, the viscosity of first fluid 431 may be significantly higher than the second fluid, or vice versa. The fluid properties may be input into the fluid simulation system as boundary conditions. For example, the inlet boundary conditions may include a velocity profile for the first fluid 431 and a second fluid 432. The velocity profile may be time varied, where the velocity of the fluid changes over a period of time. As shown in FIG. 4A, the first velocity profile 433 and the second velocity profile 434 are associated with the first fluid 431 and the second fluid 432, respectively. In some examples, the velocity profiles may be mono-exponential, gamma-variate, and power law profiles.

In FIG. 4B, model 412 illustrates the outlet boundary conditions set within the fluid simulation system at the outlet 440 of the artery-catheter model. In some examples, the outlet boundary conditions include a pressure profile 441. The pressure profile 441 may relate to the pressure of a fluid as it passes through the outlet. A Windkessel model, which considers the resistance and compliance of downstream vasculature that are not considered in the computational domain, could be prescribed at the outlet. Such models aid in improving the pressure boundary condition and subsequently enhance the accuracy of the model-predicted volume fractions.

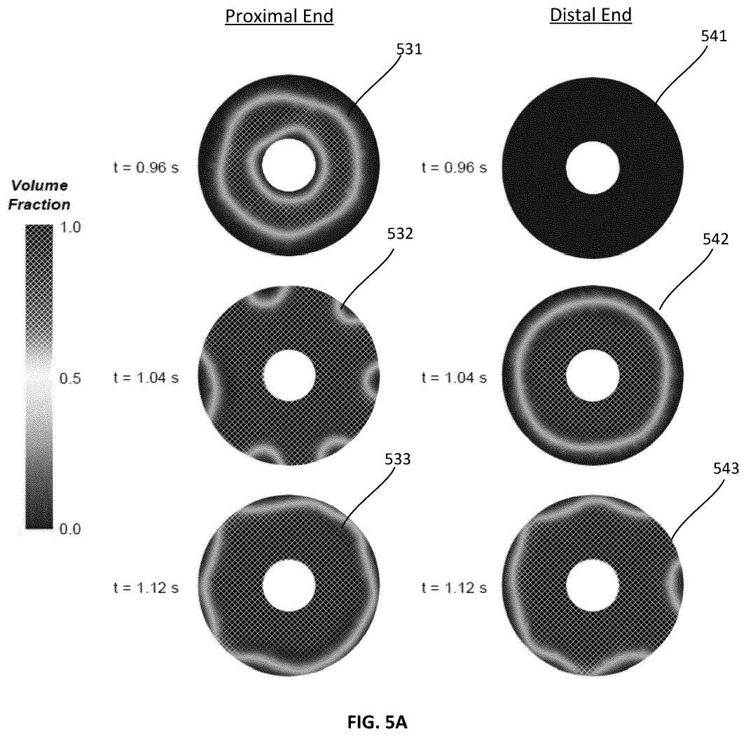

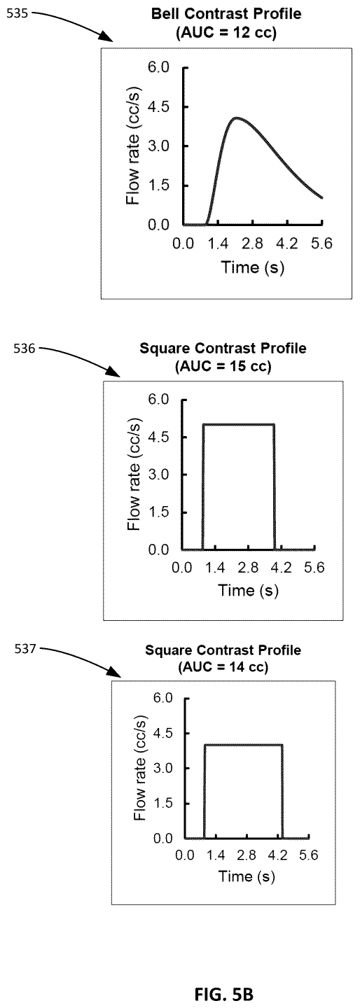

FIG. 5A-5B illustrate potential outputs of the flow simulation system. FIG. 5A illustrates the volume fraction at cross sections of the proximal end and the distal end of the ROI at various time stamps during a flow simulation. The cross sections of the proximal end and the distal end may correspond to the same time stamp during the flow simulation. For example, sections 531 and 541 correspond to 0.96 seconds into the simulation, sections 532 and 542 correspond to 1.04 seconds into the simulation, and sections 533 and 543 correspond to 1.12 seconds into the simulation.

The volume fraction may be calculated using the cross sections 531-533 and 541-543, using the scale set on the volume fraction key. As an example, according to the volume fraction key, a score of 1.0 signifies the absolute presence of a first fluid and a score of 0.0 signifies the absolute presence of a second fluid at that slice of the ROI. For example, the cross section 541 may receive a volume fraction score of 0.0, signifying that there is an absolute presence of the second fluid and the complete absence of the first fluid.

In some examples, the first fluid may be a contrast agent and the second fluid may be blood. For intravascular imaging, the best images of the artery may be taken when the contrast has substantially cleared the blood from the area to be imaged, i.e. the ROI. In some examples, a volume fraction threshold may be set for determining the optimal clearance of one fluid by the other fluid, relating to when the best images may be captured by an intravascular imaging tool. For example, a threshold of a 0.6 volume fraction score or greater may be set as optimal for intravascular imaging. The system may analyze the simulation to determine if the volume fraction threshold was sustained for a period of time. The period of time may correspond to the time needed to perform a pullback of an intravascular tool through the ROI. Where the threshold volume fraction is not sustained for the required period of time, the system may determine that a larger volume of the contrast agent is needed to obtain optimal images.

FIG. 5B illustrates example contrast flow rate profiles that may result from a flow simulation. The profiles may be a function of the flow rate of a fluid by the time measured to compete the injection of the fluid into the region of interest. The profiles may be charts with the flow rate on the y-axis and the time plotted on the x-axis. The time may be measured in seconds and the flow rate may be measured in cubic centimeters per second (cc/s). In some examples the flow rate maybe calculated by multiplying the contrast velocity by the area of the contrast inlet. The contrast velocity may vary over time. The area of the contrast inlet may be constant or fixed.

The shape of the curve of the contrast profile may reflect the manner the contrast was injected. For example, profile 535 is bell shaped, reflecting a manual injection of the contrast, and profile 536 and profile 537 are square shaped, reflecting an automated injection of the contrast. The amount of contrast injected may be determined using the plot. Amount of contrast corresponds to the area under the curve (AUC). The area under the curve is determined by integrating the start and end points of the plot. To find the area under the curve of the contrast flow rate profiles the flow rate is integrated between the limits of 0 to 5.6 seconds. This integration is performed numerically using any suitable mathematical software. In FIG. 5B, the amount of contrast injected for profile 535, 536, and 537 is 12 ccs, 15 cc, and 14 cc, respectively.

Example calculations to determine the volume of contrast may include, calculating the volume flow rate as Q=V/t where, Q is the flow rate of a volume of fluid passing through a specific location per unit of time, V is the volume of the fluid and t is the elapsed time. The flow rate is related to the average velocity of the fluid. In some examples, if the fluid moving through a vessel with a cross-sectional area A and a velocity v, the flow rate can be expressed as Q=Av where, A is the cross-sectional area of the flow and v is the average velocity of the fluid. In some examples the velocity may relate to the velocity profile of the fluid. The volume can be determined by V=Qt. In some examples, the volume can be expressed in cubic centimeters (cc).

In some examples. the optimal volume of contrast is determined by running multiple simulations involving different contrast velocity profiles. The results from the multiple simulations may be aggregated and averaged to calculate the optimal volume of contrast. In some examples, the optimal value of contrast may be determined using the volume fraction data along with the contrast profile results. For example, the system may select the results with the best visualization based on the volume fraction values, to determine the optimal volume of contrast. In some examples, the system may store the optimal value of contrast for numerous simulations and use this information to create a training network for the system. The system may use the training network to develop a machine learning model. The machine learning model may be used to calculate the optimal volume of contrast using the boundary conditions set by a user or intravascular information from image data.

FIG. 6A shows the volume fraction-time plots at a cross section of the proximal end and the distal end of the ROI during a flow simulation. Plot 632 depicts the volume fraction score of the proximal end of the ROI across the time of the injection simulation. Plot 642 depicts the volume fraction scores of the distal end of the ROI across the time of the injection simulation.

The volume fraction may also be used to determine the optimal pullback speed of an intravascular tool during a PCI or intravascular imaging procedure. Specifically, the time needed for pullback of the tool can be determined from the volume fraction-time plots 632 and 642 by evaluating the difference between the temporal value of the final and initial peaks corresponding to the proximal and distal end cross-sections, respectively. For example, peak 634 on plot 632 represents the time of the final peak of the volume fraction for the contrast agent in the artery-catheter model. Peak 634 occurs at around 4.2 seconds. Peak 644 on plot 642 represents the initial peak of the volume fraction of the contrast agent in the artery-catheter model. Peak 644 occurs around 1.9 seconds. Therefore, the ROI is most visible between these two peaks 634 and 644. Based on the volume fraction time plots 632 and 642, the system may determine the time available for pullback of an intravascular imaging tool, which is equivalent to the difference of the final peak of the proximal end and the initial peak of the distal end and whose value is about 2.3 seconds.

FIG. 6B illustrates a force time plot 652 for a fluid at the inlet of the artery-catheter model. The force needed to inject a fluid in the artery-catheter model may be calculated by multiplying the inlet area and the pressure exerted on the inlet surface. Force-flow curves can be virtually obtained when compared to mechanical contrast simulators that require extensive instrumentation. In some examples, the force required to inject the contrast is determined by multiplying the area of the contrast inlet and the corresponding model-predicted pressure. The optimal force is determined from multiple simulations involving different contrast velocity profiles. The technician would be able to control this parameter based on the optimal model-predicted value. As shown in FIG. 6B, the force time plots 652 may illustrate the amount of force used at various times during the injection of the contrast. In the plot 652, there may be peaks that represent the optimal force required to inject the contrast. In some examples, there may be a maximum peak, such as peak 654. The peak 654 may represent the optimum amount of force required to inject the contrast. In some examples, the determined optimal force may be input into the simulation or an injection system for use in a patient procedure.

The computer methodology presented here can also be used to simulate blood and contrast-agent flow in a subject-specific vascular geometry reconstructed from OCT and angiography images. Furthermore, the proposed VOF-CFD methodology can be used to evaluate possible temperature changes resulting from the mixing of two fluids by numerically solving the Navier-Stokes energy equation. This feature could be particularly useful in estimating the amount and rate at which cold saline needs to be injected for measuring blood flow using thermodilution. The output of thermodilution measurements is a temperature-time curve. The area under this curve indicates the amount of saline carried by blood per unit time. The proposed computational fluid dynamics methodology can also generate such a temperature-time curve by specifying the initial temperatures of the blood and cold saline as thermal boundary conditions at the inlet of each fluid. The spatial and temporal variation in temperature is computed by solving the Navier-Stokes energy equation in addition to the continuity and momentum equations.

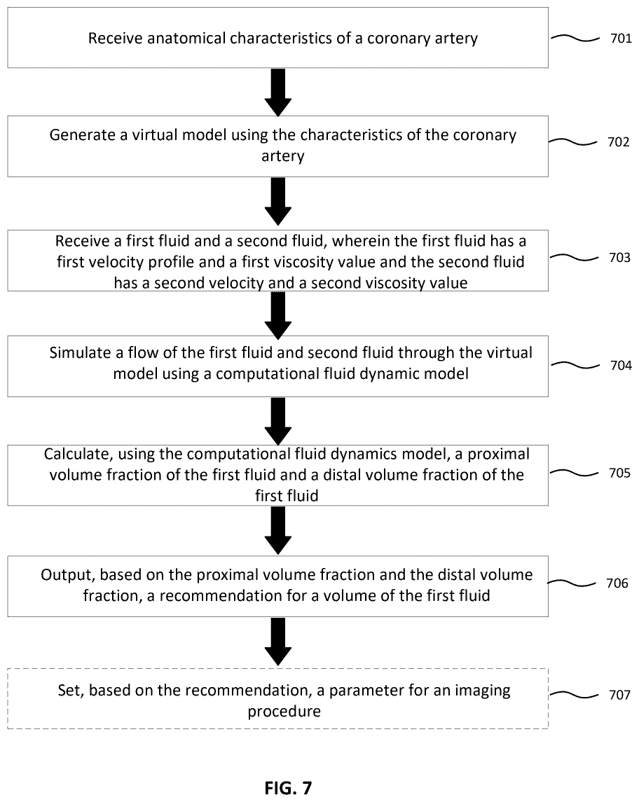

FIG. 7 illustrates an example method of outputting an optimal volume for a first fluid by generating virtual models and applying CFD methods.

In block 701, one or more processors may receive anatomical characteristics of a coronary artery. The anatomical characteristics may be patient specific. For example, the anatomical characteristics may be in the form of image data from prior intravascular imaging. In some examples, the characteristics may be an aggregate of image data for training purposes. such that the anatomical data creates an idealized artery.

In block 702, one or more processors may generate a virtual anatomical model using characteristics of the coronary artery. The virtual model may be a 3D model of the coronary artery from the image data. In some examples, the virtual artery may be a 3D model of an idealized artery. In some examples, the virtual model may include a catheter for the imaging or intravascular tool. The catheter may be represented by the virtual model. In such cases, the virtual model may be an artery-catheter assembly. The virtual model may have a region of interest. The region of interest may be a portion of the virtual model with divergent characteristics from the majority of the virtual model, such as a stenosed area. The region of interest has a proximal end and distal end. The proximal end may be closer to an inlet of the virtual model, whereas the distal end may be closer to the outlet of the virtual model. The direction of the flow in the virtual model is from the proximal end to the distal end.

In block 703, one or more processors may receive a first fluid and a second fluid. The first fluid may have a first velocity profile and a first constant viscosity value. The second fluid may have a second velocity profile and a second constant viscosity value. In some examples, the first fluid may be a contrast agent and the second fluid may be blood within the artery. The fluids may be concentrically aligned within the virtual model such that the contrast agent is concentrically within the blood.

In block 704, one or more processors may simulate a flow of the first fluid and the second fluid through the virtual anatomical model using a computational fluid dynamics model. The computational fluid dynamics model may have various boundary conditions. These boundary conditions may be set at specific aspects of the virtual model, such as the inlet, the outlet, and the wall. The first and second velocity profiles may be set at the inlet of the virtual model. the first and second viscosity constant values may be applied to the first and second fluids directly.

In block 705, one or more processors may calculate, using the computational fluid dynamics model, a proximal volume fraction of the first fluid and a distal volume fraction of the first fluid. The proximal volume fraction of the first fluid may be collected from a slice in the proximal end of the region of interest. The distal fraction of the first fluid may be collected from a slice in the distal end of the region of interest. The volume fraction may be plotted across the time for an injection of the first fluid to clear the region of interest. In some examples, a threshold may be set for the volume fraction to determine optimal imaging time. In some examples, a volume fraction threshold may be set for determining the optimal clearance of one fluid by the other fluid, relating to when the best images may be captured by an intravascular imaging tool.

In block 706, one or more processors may output, based on the proximal volume fraction and the distal volume fraction, a recommendation for a volume of the first fluid. The volume of fluid may relate to the amount of contrast required to clear the blood for the imaging procedure.

In block 707, one or more processors may optionally set, based on the recommendation, a parameter for an imaging procedure. In some examples, the output may be entered into an imaging system to be automatically applied to an intravascular imaging procedure. In some examples, the output may relate to the time available for pullback of an intravascular imaging tool or the injection force necessary for the first fluid, i.e. the contrast agent.

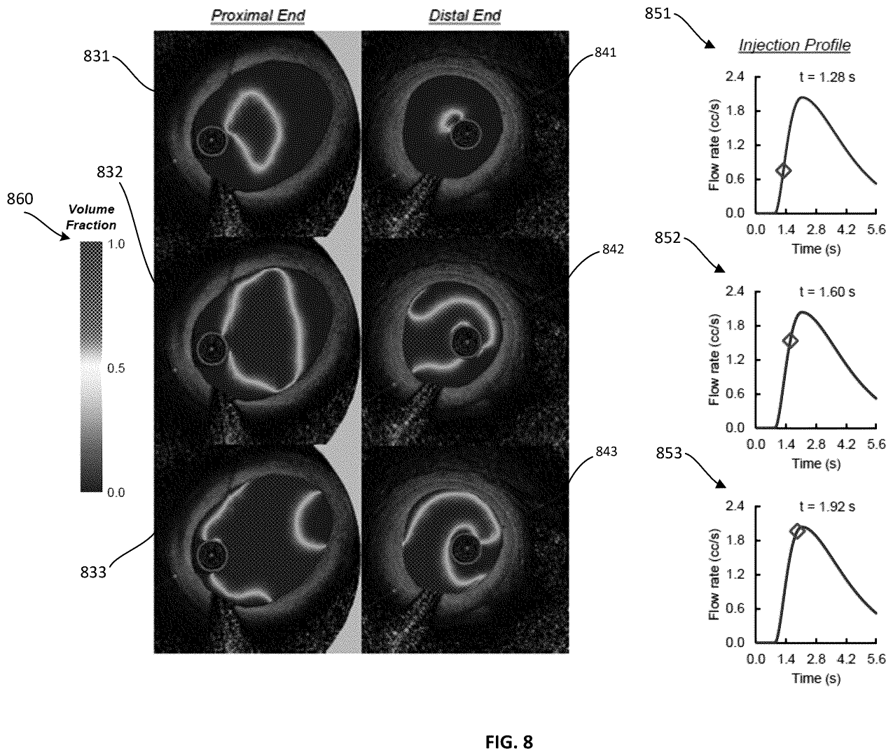

As discussed herein, the computer methodology presented within the current disclosure can be used to simulate blood and contrast-agent flow in a subject-specific vascular geometry reconstructed from OCT and angiography images. FIGS. 8-11 illustrate potential outputs of the flow simulation system using subject-specific vascular geometry. For example, FIG. 8 provides volume fraction results at cross-sections of the proximal end and the distal end of the ROI at various time stamps during a subject-specific flow simulation. The cross-sections of the proximal end and the distal end may correspond to the same time stamp during the flow simulation. For example, sections 831 and 841 correspond to 1.28 seconds into the simulation, as indicated by injection profile graph 851. In addition, sections 832 and 842 correspond to 1.6 seconds into the simulation, as indicated by injection profile graph 852, and cross-sections 833 and 843 correspond to 1.92 seconds into the simulation, as indicated by injection profile graph 853.

The injection profile graphs 851-853 show a bell-shaped injection profile for a subject with a coronary artery having a moderate stenosis. The volume fraction of the injected contrast agent can be determined for the proximal and distal ends using cross-sections 831-833 and 841-843, respectively, using the volume fraction key 860, with color volume fraction results for the CFD overlaid on each OCT image for ease of visualization.

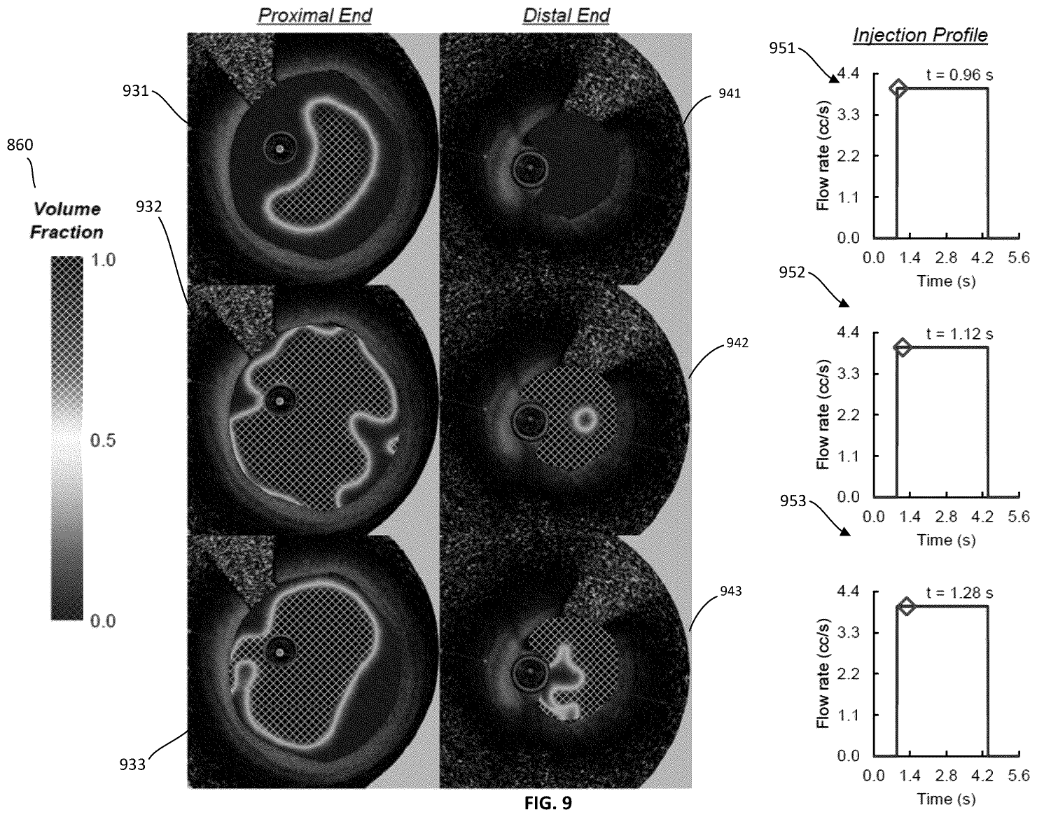

FIG. 9 provides subject-specific volume fraction results at cross-sections of the proximal end and distal end of the ROI using a square-shaped injection profile. Sections 931 and 941 correspond to 0.96 seconds into the simulation, as indicated by injection profile graph 951. In addition, sections 932 and 942 correspond to 1.12 seconds into the simulation, as indicated by injection profile graph 952, and sections 933 and 943 correspond to 1.28 seconds into the simulation, as indicated by injection profile graph 953. The volume fraction of the injected contrast agent can be determined for the proximal and distal ends using cross-sections 931-933 and 941-943, respectively, using the volume fraction key 860, with color volume fraction results for the CFD overlaid on each OCT image for ease of visualization.

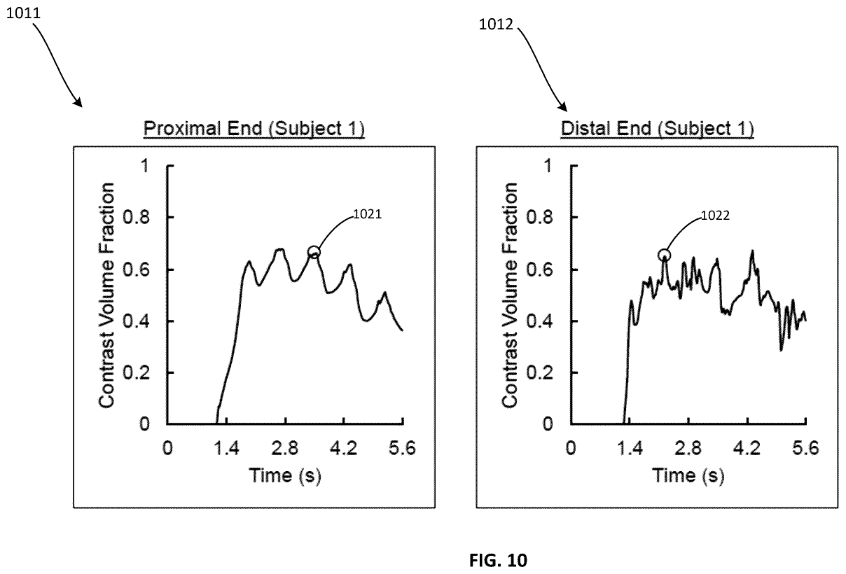

FIG. 10 are graphs 1011 and 1012 of time varying contrast-agent volume fractions for the proximal and distal cross-sections of a subject-specific artery having a moderate stenosis. Graphs 1011 and 1012, as well as the data used to generate graphs 1011 and 1012, may be provided as an output of the system in which a subject-specific simulation is performed. Indications 1021 and 1022 of graphs 1011 and 1012, respectively, serve as peak markers for indicating the time and the peak contrast volume fraction within the simulation. Indication 1021 may be configured to indicate the last peak within the proximal end graph 1011, while indication 1022 may be configured to indicate the first peak within the distal end graph 1012. Based on the volume fraction time plots 1011 and 1012, the system may determine the time available for pullback of an intravascular imaging tool, which is equivalent to the difference of the final peak of the proximal end and the initial peak of the distal end and whose value for graphs 1011 and 1012 is about 1.3 seconds.

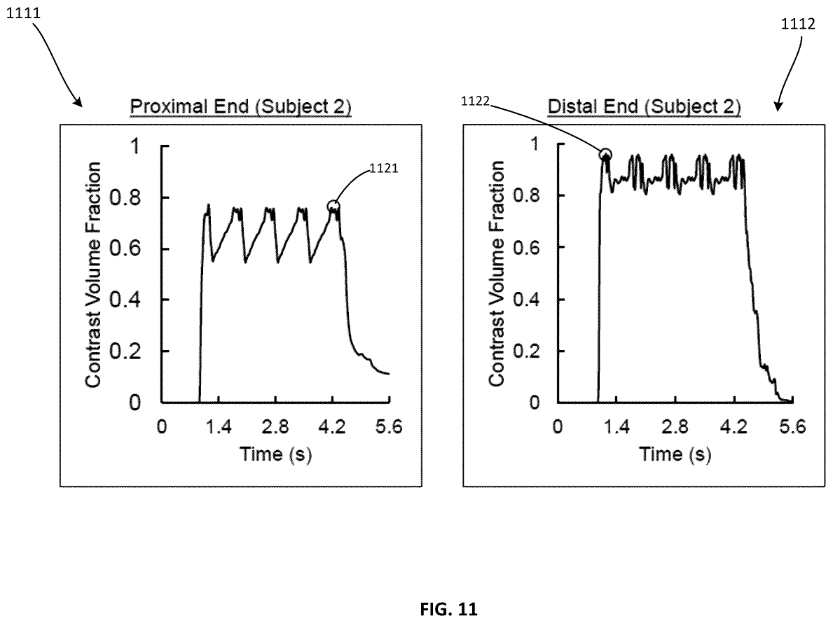

FIG. 11 are graphs 1111 and 1112 of time varying contrast-agent volume fractions for the proximal and distal cross-sections of a subject-specific artery having a mild stenosis. Graphs 1111 and 1112, as well as the data used to generate graphs 1111 and 1112, may be provided as an output of the system in which a subject-specific simulation is performed. Indications 1121 and 1122 of graphs 1111 and 1112, respectively, serve as peak markers for indicating the time and the peak contrast volume fraction within the simulation. Indication 1121 may be configured to indicate the last peak within the proximal end graph 1111, while indication 1122 may be configured to indicate the first peak within the distal end graph 1112. Based on the volume fraction time plots 1111 and 1112, the system may determine the time available for pullback of an intravascular imaging tool, which is equivalent to the difference of the final peak of the proximal end and the initial peak of the distal end and whose value for graphs 1111 and 1112 is about 3.1 seconds.

In order to reconstruct the 3-D geometry of the subject-specific coronary artery a 3-D stack of OCT images can be converted from color to binary format and input into an image-processor. In addition, a segmentation algorithm may be used to identify the coronary artery within the binary images. The resulting geometry may be smoothed to remove unwanted surface artifacts. In addition, the determined geometry may be converted to a stereolithography (STL) format. The STL geometry can be processed by the disclosed system to include flow extensions and so as to integrate the 3-D geometries of the guiding and OCT catheters using Boolean operations, as disclosed in connection with other models herein.

In addition, the subject-specific artery model may be discretized using polyhedral elements to create a CFD mesh of the subject-specific artery, and boundary conditions may be applied to the model in connection with simulation of fluid flow. For example, boundary conditions may include a condition for no slip at the rigid walls, time-varying pressure at the outlet, and time-varying velocity at the fluid inlets. Thermophysical properties may also be assigned and blood-contrast agent flow may be simulated using a VOF method described herein.

Machine learning may also be used to generate model predictions, so as to allow for shifts in computational costs and allow for increased efficiency of some models. Specifically, deep-learning neural networks (DNNs) can be used to reduce calculation time with respect to CFD simulations of blood-contrast agent flow in subject-specific artery geometries. The model of the artery geometry and contrast-injection profile data can serve as inputs to the DNN, with the DNN providing an output of spatially and temporally varying contrast-agent volume fractions. For example, combinations of 100 artery shapes and 10 injection profiles would result in 1,000 datasets for input into the DNN. These datasets may be augmented by dividing the datasets into multiple time points and/or by generating synthetic artery geometries using statistical shape modeling. While model results can be determined with other parameters, such as using generic blood-velocity and blood-pressure profiles, being set values, these parameters may also be varied and serve as additional inputs to the DNN.

The aspects, embodiments, features, and examples of the disclosure are to be considered illustrative in all respects and are not intended to limit the disclosure, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise. As used herein, the term “about” refers to a ±10% variation from the nominal value. All numerical values and ranges disclosed herein are deemed to include “about” before each value.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method, comprising:
receiving, by one or more processors, anatomical characteristics of a coronary artery;
generating, by the one or more processors based on the anatomical characteristics, a virtual model of the coronary artery, wherein the virtual model includes a region of interest of the coronary artery, the region of interest comprising a proximal end and a distal end;
receiving, by the one or more processors, a selection of a first fluid and a second fluid, wherein the first fluid has a first velocity profile and a first viscosity value, wherein the second fluid has a second velocity profile and a second viscosity value;
simulating, by the one or more processors using a computational fluid dynamics model using the first viscosity value and second viscosity value, a simultaneous flow of the first fluid and the second fluid through the virtual model;
wherein the computational fluid dynamics model has boundary conditions of the simulation include at least the first velocity profile of the first fluid and the second velocity profile of the second fluid;
calculating, by the one or more processors using the computational fluid dynamics model based on the first velocity profile and the flow of the first fluid, a flow rate profile of the first fluid, wherein the flow rate profiles are a function of the flow of the first fluid within the virtual model and time; and
outputting, by the one or more processors based on the flow rate profile, a recommendation for a volume of the first fluid.

2. The method of claim 1, wherein the recommendation for the volume of the first fluid is a recommendation for recommended the volume of the first fluid to be injected into the coronary artery.

3. The method of claim 2, further comprising setting, based on the recommendation, a parameter on a device arranged to automatically control an injection of the first fluid into the coronary artery.

4. The method of claim 1, further comprising:
calculating, by the one or more processors using the computational fluid dynamics model, a proximal volume fraction of the first fluid at the proximal end and a distal volume fraction of the first fluid at the distal end;
calculating, by the one or more processors, a time for the first fluid to flow from the proximal end to the distal end of the virtual model; and
calculating, by the one or more processors using the computational fluid dynamics model, a time frame available for a pullback of an imaging tool,
wherein the time frame is derived from a comparison of a first function of the proximal volume fraction by the measured time and a second function of the distal volume fraction by the measured time.

5. The method of claim 4, further comprising
setting, by the one or more processors based on the time available for the pullback, a pullback time for the imaging tool.

6. The method of claim 1, wherein the recommendation for the volume of the first fluid corresponds to an area under a curve of the flow rate profile.

7. The method of claim 1, further comprising
calculating, by the one or more processors using the computational fluid dynamics model, an injection profile for the first fluid derived from a function of injection force of the first fluid by time.

8. The method of claim 1, wherein the virtual model has a first fluid inlet, a second fluid inlet, an outlet and a wall.

9. The method of claim 8, wherein the boundary conditions further include no slip condition applied to the wall of the virtual model.

10. The method of claim 8, wherein the boundary conditions further include a pressure profile applied to the outlet of the virtual model.

11. The method of claim 1, wherein the anatomical characteristics are determined from intravascular data collected from an intravascular imaging device, wherein the intravascular imaging device is an optical coherence tomography probe or an intravascular ultrasound probe.

12. A system comprising:
one or more processors, the one or more processors configured to:
receive anatomical characteristics of a coronary artery;
generate, based on the anatomical characteristics of the coronary artery, a virtual model, wherein the virtual model includes a region of interest comprising a proximal end and a distal end;
receive a first fluid and a second fluid, wherein the first fluid has a first velocity profile and a first constant viscosity value and the second fluid has a second velocity profile and a second constant viscosity value;
simulate, using a computational fluid dynamics model using the first viscosity value and second viscosity value, a flow of the first fluid and the second fluid through the virtual model;
wherein the computational fluid dynamics model has boundary conditions including at least the first velocity profile and the second velocity profile;
calculate, using the computational fluid dynamics model, a proximal volume fraction of the first fluid at the proximal end and a distal volume fraction of the first fluid at the distal end;
calculate, using the computational fluid dynamics model based on the first velocity profile and the flow of the first fluid, a flow rate profile of the first fluid; and
output, based on the proximal volume fraction, the distal volume fraction, and the flow rate profile, a recommendation for a volume of the first fluid.

13. The system of claim 12, wherein the one or more processors are further configured to set, based on the recommendation, a parameter for the volume of the first fluid to be injected into the coronary artery.

14. The system of claim 12, wherein the one or more processors are further configured to:
measure a time for the first fluid to flow from the proximal end to the distal end of the virtual model; and
calculate, using the computational fluid dynamics model, a time frame available for a pullback of an imaging tool,
wherein the time frame is derived from a comparison of a first function of the proximal volume fraction by the measured time and a second function of the distal volume fraction by the measured time.

15. A system of claim 14, wherein the one or more processors are further configured to:
set, based on the time frame available for the pullback, a pullback time for the imaging tool.

16. A system of claim 12, wherein the recommendation for the volume of the first fluid corresponds to an area under a curve of the flow rate profile.

17. A system of claim 12, wherein the one or more processors are further configured to:
calculate, using the computational fluid dynamics model, an injection profile for the first fluid derived from a function of injection force of the first fluid by time.

18. A system of claim 12, wherein the virtual model has a first fluid inlet, a second fluid inlet, an outlet and a wall.

19. A system of claim 18, wherein the boundary conditions further include no slip condition applied to the wall of the virtual model.

20. A system of claim 17, wherein the boundary conditions further include a pressure profile applied to the outlet of the virtual model.
